(19) United States
(12) Patent Application Publication
(10) Pub. No.: US 2013/0077756 A1
(43) Pub. Date: Mar. 28, 2013

(54) IMAGING METHOD AND APPARATUS FOR DISPLAYING VESSELS OR ORGANS IN AN AREA OF A PATIENT UNDER EXAMINATION

(71) Applicant: SIEMENS AKTIENGESELLSCHAFT, Muenchen (DE)

(72) Inventor: Matthias Saar, Altendorf (DE)

(73) Assignee: SIEMENS AKTIENGESELLSCHAFT, MUENCHEN (DE)

(21) Appl. No.: 13/626,124

(22) Filed: Sep. 25, 2012

(30) Foreign Application Priority Data
Sep. 26, 2011 (DE) 102011083408.7

(51) Int. Cl. H05G 1/64; A61B 5/029

(52) U.S. Cl. 378/98.12; 600/526

(57) ABSTRACT

An imaging method and apparatus for displaying a target object, such as one or a plurality of blood vessels and/or an organ in an area of a patient under examination, is provided. The examination can be a medical intervention. At least one recorded fluoroscopic image of the area under examination is recorded by an X-ray system. At least one up-to-date reconstructed 3D radar image is generated from signals detected a radar receiver. The target object is identified in the fluoroscopic image and in the radar image. The radar image with the fluoroscopic image is recorded with the aid of the result of the identification. The radar image and the fluoroscopic image are combined. The combined image is reproduced on a display device.

### CROSS REFERENCE TO RELATED APPLICATIONS

This application claims priority of German application No. 10 2011 083 408.7 filed Sep. 26, 2011, which is incorporated by reference herein in its entirety.

### FIELD OF INVENTION

The application relates to an imaging method and apparatus for displaying at least one target object, such as one or a plurality of blood vessels and/or an organ in an area of a patient under examination, such as during a medical intervention.

### BACKGROUND OF INVENTION

Two- and three-dimensional digital subtraction-rotational angiography (2D or 3D DSA rotational angiography) is a standard method used for assessing the vascular anatomy before and during medical interventions. In the case of digital subtraction angiography (DSA), after the generation of mask images, images without contrast medium, and filler images, images with contrast medium, these are subtracted from one another so that only the time-related changes caused by the contrast medium, which represent the vessels, are obtained.

A C-arm X-ray system for digital subtraction angiography of the type illustrated by way of an example in FIG. 1, has for example a C-arm 2 mounted in a rotatable manner on a pedestal in the form of a six-axis industrial or articulated-arm robot 1, at the ends of which are mounted an X-ray radiation source, for example an X-ray emitter 3 with X-ray tube and collimator, and an X-ray image detector 4 as an imaging unit.

By rotating it about a center of rotation between the X-ray emitter 3 and the X-ray detector 4, for example, the C-arm 2 can be adjusted to any spatial position by an articulated-arm robot 1, such asably having six axes of rotation and six degrees of freedom. The articulated-arm robot 1 has a basic frame which, for example, is mounted on the floor in a stationary manner and to which is attached a carousel that is rotatable about a first axis of rotation. A robot rocker beam is swivel-mounted on the carousel about a second axis of rotation and to which a robot arm is attached in a rotatable manner about a third axis of rotation. A robot hand is mounted at the end of the robot arm in a rotatable manner about a fourth axis of rotation. The robot hand has a mounting element for the C-arm 2, which can swivel about a fifth axis of rotation and can be rotated about a sixth axis of rotation that is perpendicular to said fifth axis of rotation.

Realization of the X-ray diagnostic apparatus does not rely on the industrial robot. Conventional C-arm devices can also be employed.

The X-ray image detector 4 can be a rectangular or square, flat semiconductor detector which is developed from amorphous silicon (a-Si). However, integrating and possibly metering CMOS detectors can also be used.

A patient table 5 for recording the heart of a patient 6 as the subject of examination, for example, is located in the beam of the X-ray emitter 3. A system control unit 7 with a display system 8, which receives and processes the video signals of the X-ray image detector 4 (operator control elements, for example, are not shown), is connected to the X-ray diagnostic apparatus. The X-ray images can then be viewed on a monitor 9. In one embodiment, a suspended monitor arrangement 13 with a first display 14 and at least one further display 15 can be attached to the ceiling.

Two C-arms are being increasingly used in radiology. These are so-called biplane systems.

In X-ray diagnostics, a screened organ or blood vessel is represented in two dimensions. A 3D representation is possible by rotating the C-arm about the organ or vessel, with simultaneous sequential recording. As a result, dependent upon image frequency and rotational speed, several hundred two-dimensional X-ray images are created, which can then be converted into 3D images.

It is possible with the aid of ultra wideband (UWB) radar to compute the third dimension—even with an individual X-ray image. As a result, the patient is exposed to a much lower X-ray dosage.

Other features of combining X-ray apparatus with UWB radar:
Contactless patient monitoring and anti-collision system,
Triggering (starting or initiation) of the X-ray recording in accordance with the flow of contrast medium without fluoroscopy or without X-ray radiation, and
Determination of the heart pumping volume without X-ray radiation.

As already mentioned in the introduction, 3D X-ray images are generated by rotating the X-ray apparatus around the patient. By doing this, X-ray images are generated in each angle of rotation and converted into 3D in a computer, for example the said display system 8.

A patient examination is realized by connecting the patient to an ECG unit, for example. Initiation of the X-ray radiation normally occurs after a time delay; the doctor injects the contrast medium and from his experience knows approximately how rapidly the contrast medium spreads. He then activates the X-ray radiation. A series of X-ray images is then initiated by the left ventricle of the heart, for example; the two heart phases of interest are selected and the pumping volume is calculated.

### SUMMARY OF INVENTION

The application is based on the problem of improving the representation of the target object in the area under examination, for example vessels, in accordance with the method or the medical apparatus mentioned in the introduction.

The object of the application is achieved by the subject matter of the independent claims. Developments of the application are revealed in the features of the dependent claims.

Due to the disclosed combination of the UWB radar and the X-ray installation it is possible to determine the movements of the heart and the coronary vessels in the depth of the body in a contact-free manner and to combine the radar image with an X-ray image, which results in a 3D image that has been generated at a low dosage. The 3D image is reproduced on an indicating device, for example a display or monitor.

Compared to the prior art, the combination of UWB radar and X-ray angiography provides an improved, up-to-date display of the blood vessels in the area under examination and exposes the patient to a lower level of radiation.

Patient monitoring is likewise possible in a contact-free manner. No patient monitors (for example ECG) are necessary in the treatment room. This results in an extended anti-collision system around the patient. The triggering of the X-ray apparatus can be controlled by the flow of contrast medium in the patient. The pumping volume of the heart can be measured and calculated without radiation.

### BRIEF DESCRIPTION OF THE DRAWINGS

Embodiments of the application with developments according to the features of the dependent claims are explained in more detail with the aid of the following drawing, without being restricted to them.

FIG. 1 shows by way of an example an X-ray system, configured as an X-ray C-arm system with an industrial robot as the supporting device, and

FIG. 2 shows a disclosed arrangement for combining X-ray diagnostics with ultra wideband radar.

### DETAILED DESCRIPTION OF INVENTION

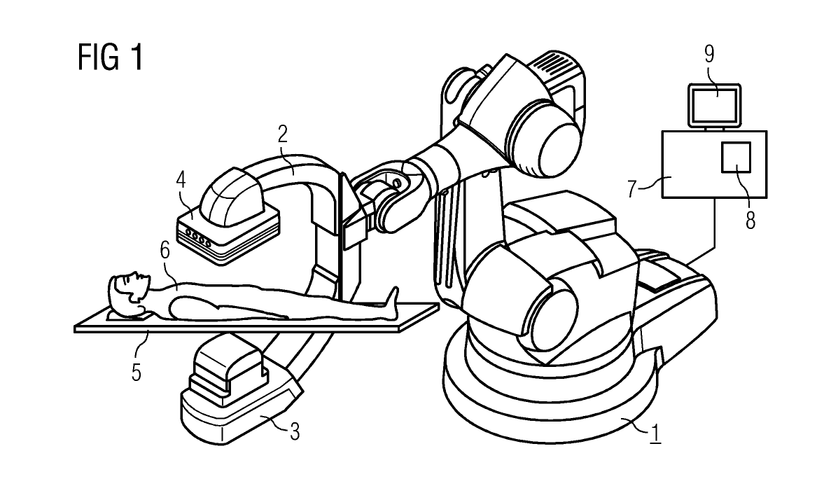

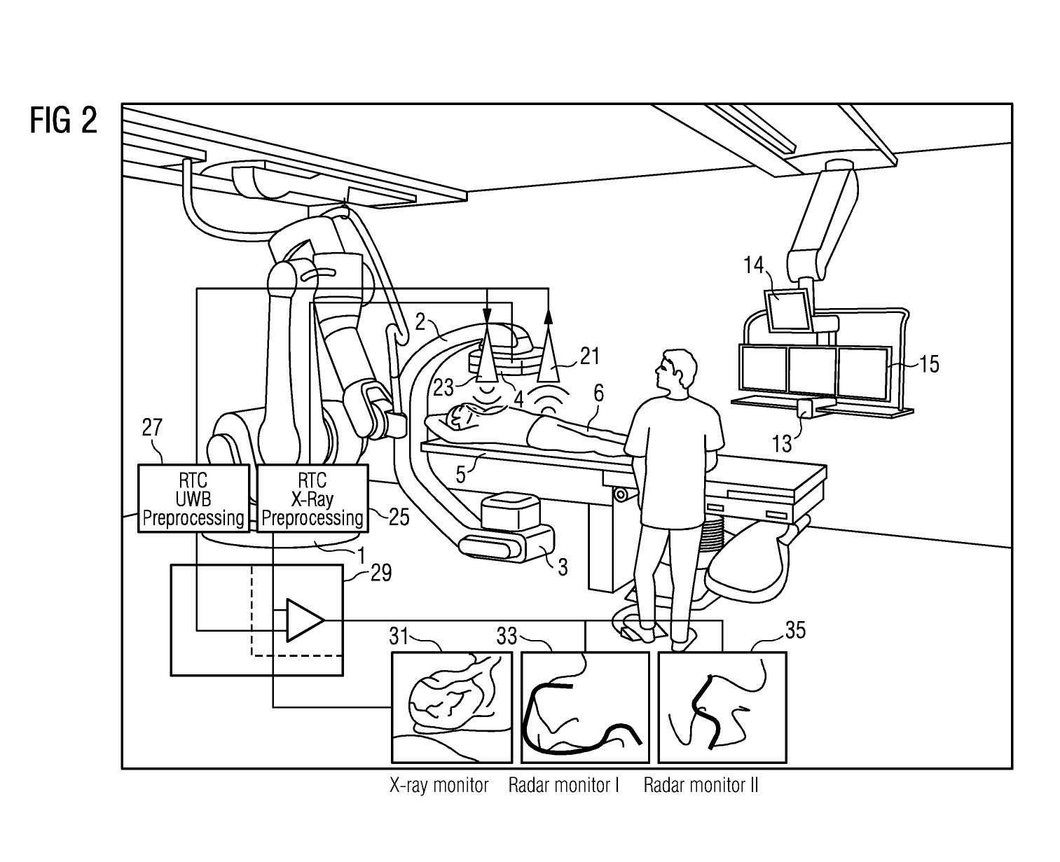

FIG. 2 shows by way of an example a disclosed arrangement which includes the components of the system described in FIG. 1 and is additionally configured around components for the ultra wideband radar. Included among others are UWB sensors 21 and UWB transmitters 23 and components for real time control (RTC) of X-ray processing 25 and for RTC UWB processing 27, which are coupled together. This is denoted by the components of an electronic evaluator 29 which can be integrated in a computer, for example an image computer 8, it being possible, as a result, for images to be represented on at least one X-ray monitor 31 (for example display 14) as well as on at least one radar monitor I 33 or II 35 (for example display 15).

According to the application, the procedure is as follows:
1. Detection of movement in the heart and coronary blood vessels.

UWB is a radio modulation technique based on the transmission and reception of pulses of very short duration (often less than one nano second (<10⁻⁹ s) with a very wide bandwidth. The signals reflected from different depths of the body are detected with a receiving antenna or receiving sensor. Due to the heart beat and the resulting movements of the coronary blood vessels, the boundary layers of the organ are displaced and deformed, thus influencing the measured signal. Measurement data can be obtained from these anatomical movements and the movement of the organ and the coronary movement can be reconstructed with respect to location and time.

If the patient is irradiated with low-power (<1 mW) wideband electromagnetic pulses from different directions, then these penetrate to different depths of the body and are partially reflected at successive boundary layers of the various types of tissue.

Since the various types of human tissue have typical absorption and reflection characteristics, organ movements such as heartbeat and the movements of the coronary vessels can be precisely detected by ultra wideband radar systems.

Signals (see RTC UWB preprocessing 27) which are analogous to the movement of the heart/of the coronary vessels and enable a 3D reconstruction of the moving heart, are generated from the receivers of the UWB radar system in an electronic evaluator with a computer. This 3D data set can now be fed to the image computer 8 of the X-ray system and combined with the X-ray image, for example as in the Siemens AG “Axiom Ards” X-ray system and the “AXIS” image computer 8.

The associated radar image (radar images are three-dimensional) is assigned in the image computer to each X-ray or fluoroscopic image (these are two-dimensional). A new 3D image that combines the features of the X-ray image (higher resolution) with the features of the UWB radar system (3D representation without radiation burden) is produced by combining the X-ray image with the UWB radar image.
2. Patient monitoring

Vital patient functions, such as breathing or heart rhythm, can be monitored with the aid of the UWB radar in a contact-free manner. Possible patient panic states can also be immediately detected and appropriate measures initiated.
3. Collision monitoring

Furthermore, it is possible with the aid of the UWB radar to prevent unintentional contact with sterile devices in the examination room and possibly to trigger an alarm.

The movements of the patient table and the X-ray apparatus can also be monitored with the system. Movement can be stopped and/or an alarm triggered as soon as a patient, an operator or a device is located in the collision zone.
4. Triggering the X-ray radiation

As the various types of human tissue have typical absorption and reflection characteristics, blood vessels can be accurately detected and displayed by the four ultra wideband radar systems. The flow of blood or contrast medium in the vessels can be measured by the so-called Doppler effect. Consequently, it is possible to trigger the X-ray radiation only when the contrast medium has reached the appropriate position in the vessel.
5. Determination of the ejection fraction without X-ray radiation

The pumping volume of the left ventricle of the heart can be determined. (EF=ejection fraction).

The heart phases of interest are the end diastole (ED) and the end systole (ES). The volumes in the respective heart phase can be determined and the ejection fraction (EF) calculated with the UWB radar system.

The ejection fraction corresponds to the ratio in percentage of EDV and ESV to EDV and is expressed mathematically as:

100% ×(EDV−ESV)/ESV,

where EDV (ml) is the volume of the ventricle in the ED phase and ESV (ml) is the volume of the ventricle in the ES phase.

1. A method for displaying a target object in an area of a patient under examination, comprising the steps of:
recording a fluoroscopic image of the area under examination by an X-ray system;
generating a up-to-date reconstructed 3D radar image from signals detected by a radar receiver;
identifying the target object in the fluoroscopic image and in the radar image;
recording the radar image with the fluoroscopic image based on the identification; and
combing the radar image and the fluoroscopic image to generate a combined image.

2. The method as claimed in claim 1, wherein the combined image is a 3D image and is reproduced on a display device.

2. The method as claimed in claim 2, wherein the 3D reproduction of the combined image is continuously performed.

3. The method as claimed in claim 1, wherein the steps of the method are repeated in a selectable time interval until completion of the method.

4. The method as claimed in claim 1, wherein a plurality of fluoroscopic images is continuously recorded.

5. The method as claimed in claim 1, wherein flow of blood and/or contrast medium in a vessel of the area under examination is measured by radar Doppler effect.

6. The method as claimed in claim 5, wherein recording of the fluoroscopic image is triggered when the blood and/or contrast medium reaches an appropriate position in the vessel

7. An imaging medical apparatus, comprising:
an X-ray system for recording a fluoroscopic image of an area under examination;
a radar transmitter for transmitting signals;
a radar receiver for detecting the transmitted signals;
a radar image processing unit for generating a reconstructed 3D radar image from the detected signals;
an image storage unit for buffer storage of the fluoroscopic image and/or of the reconstructed 3D image;
a first image processing unit for identifying a target object in the reconstructed 3D image;
a second image processing unit for identifying the target object in the fluoroscopic image;
a recording device for recording the reconstructed 3D image and the fluoroscopic image based on the identification;
a combining device for combining the reconstructed 3D image and the fluoroscopic image; and
a 3D display device for displaying the combined image.

8. The apparatus as claimed in claim 7, further comprising a measuring device for measuring flow of blood and/or contrast medium in a vessel of the area under examination by radar Doppler effect.

9. The apparatus as claimed in claim 8, wherein the X-ray system is trigger for recording the fluoroscopic image when the blood and/or contrast medium reaches an appropriate position in the vessel.

10. The apparatus as claimed in claim 7, further comprising a patient table and a movement of the patient table is monitored.

11. The apparatus as claimed in claim 10, wherein the movement of the patient table is stopped and/or an alarm is triggered when a patient, an operator or a device is located in a collision zone of the apparatus.

12. The apparatus as claimed in claim 7, wherein the apparatus is used for determining a pumping volume of a left ventricle of a heart of a patient.
